(19) United States
(12) Patent Application Publication
(10) Pub. No.: US 2008/0167479 A1
(43) Pub. Date: Jul. 10, 2008

(54) PROCESS FOR PREPARING VILDAGLIPTIN

(75) Inventors: Stephen Winter, Santa Coloma de cervello (ES); Jordi Bosch, Girona (ES); Jordi Puig Serrano, Girona (ES); Jose Javier Soto, Alicante (ES)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Medichem, S.A., Barcelona (ES)

(21) Appl. No.: 11/972,329

(22) Filed: Jan. 10, 2008

Related U.S. Application Data
(60) Provisional application No. 60/879,670, filed on Jan. 10, 2007.

(51) Int. Cl. C07D 207/16; C07D 401/12; G01N 30/00

(52) U.S. Cl. 548/523; 548/528; 73/61.52

(57) ABSTRACT

The present invention relates to a process for preparing vildagliptin of formula (I) with high chemical and enantiomeric purity and compositions comprising vildagliptin. In addition, the present invention relates to (2S,2′S)-1,1′-[[(3-hydroxytricyclo[3.3.1.1^{3,7}]dec-1-yl)imino]bis(1-oxo-2,1-ethanediyl)]di(2-pyrrolidinecarbonitrile) of formula (II), processes for preparing, and compositions comprising a compound for formula (II). Furthermore, the invention relates to processes for determining the purity of vildagliptin using a compound of formula (II).

### CROSS-REFERENCE TO RELATED APPLICATIONS

This patent application claims the benefit of U.S. Provisional Patent Application No. 60/879,670, filed Jan. 10, 2007, which is incorporated by reference.

### BACKGROUND OF THE INVENTION

Vildagliptin is an active pharmaceutical substance with an empirical formula of C₁₇H₂₅N₃O₂ and a molecular weight of 303.40 g/mol. Vildagliptin is the international common accepted name for (2S)-1-[[(3-hydroxytricyclo[3.3.1.1^{3,7}]dec-1-yl)amino]acetyl]-2-pyrrolidine carbonitrile and has the structure of formula (I).

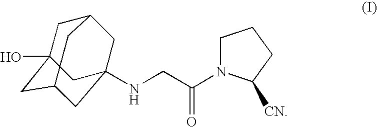

Vildagliptin is a dipeptidyl peptidase IV (DPP-IV) inhibitor and is disclosed in U.S. Pat. No. 6,166,063 (“the '063 patent”), the disclosure of which is incorporated herein by reference. The '063 patent discloses a synthesis of vildagliptin using the synthetic process represented in Scheme 1.

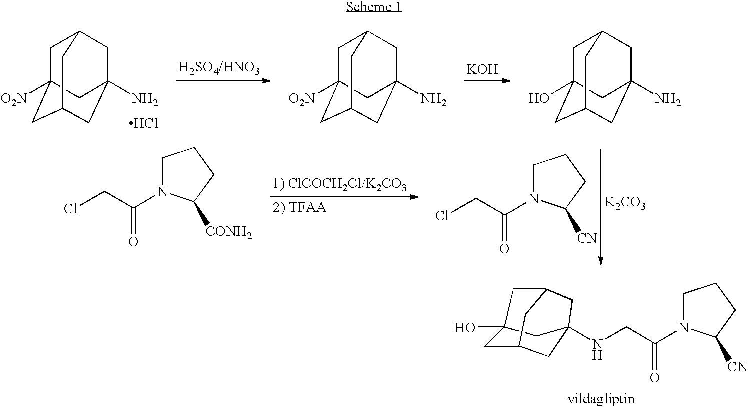

Vildagliptin can exist as the (2S) and (2R) enantiomers. The stereoisomer with the desired biological activity is the (2S) enantiomer. Accordingly, it is desirable to synthesize (2S)-vildagliptin with high stereochemical purity. A process that yields vildagliptin with a high enantiomeric purity is disclosed in International Patent Publication WO 2004/092127, the disclosure of which is incorporated herein by reference. This reference discloses compositions containing from 95% to 99.99% of (2S)-vildagliptin.

However, there remains a need for a process for preparing vildagliptin with high chemical and enantiomeric purity. The invention provides such a process. These and other advantages of the invention as well as additional features will be apparent from the description of the invention provided herein.

### BRIEF SUMMARY OF THE INVENTION

The invention provides (2S)-1-[N-(3-hydroxytricyclo[3.3.1.1^{3,7}]dec-1-yl)glycyl]-2-pyrrolidinecarbonitrile of formula (I) (i.e., vildagliptin) with high chemical and enantiomeric purity:

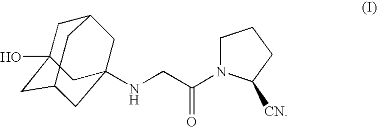

The invention also provides (2S,2′S)-1,1′-[[(3-hydroxytricyclo[3.3.1.1^{3,7}]dec-1-1-yl)imino]bis(1-oxo-2,1-ethanediyl)]di(2-pyrrolidinecarbonitrile) of formula (II):

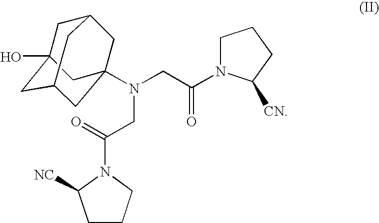

The invention further provides a process for preparing vildagliptin and the compound of formula (II) comprising reacting a compound of formula (III):

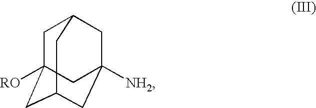

wherein R is hydrogen or an oxygen protecting group with a compound of formula (IV):

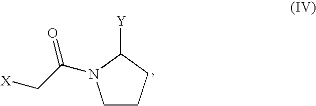

wherein X is halogen and Y is —C(O)NH₂ or —CN.

In other embodiments, the invention provides a method for determining the purity of vildagliptin comprising the use of a compound of formula (II) as a reference marker.

In other embodiments, the invention provides compositions comprising vildagliptin.

### BRIEF DESCRIPTION OF THE DRAWING(S)

FIG. 1 is a powder X-ray diffractogram of vildagliptin prepared according to Example 5.

### DETAILED DESCRIPTION OF THE INVENTION

The invention provides a process for preparing (2S)-1-[N-(3-hydroxytricyclo[3.3.1.1^{3,7}]dec-1-yl)glycyl]-2-pyrrolidinecarbonitrile (i.e., vildagliptin) of formula (I):

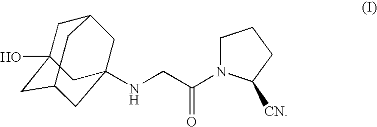

The invention also provides (2S,2′S)-1,1′-[[(3-hydroxytricyclo[3.3.1.1^{3,7}]dec-1-yl)imino]bis(1-oxo-2,1-ethanediyl)]di(2-pyrrolidinecarbonitrile) of formula (II):

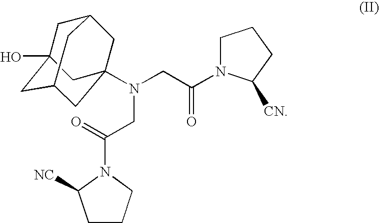

In preferred embodiments, the compound of formula (II) is used as a reference marker for the preparation of vildagliptin.

In accordance with the invention, the process for making (2S)-1-[N-(3-hydroxytricyclo[3.3.1.1^{3,7}]dec-1-yl)glycyl]-2-pyrrolidinecarbonitrile (i.e., vildagliptin) comprises reacting a compound of formula (III):

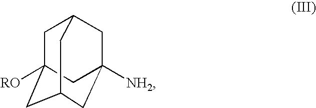

wherein R is hydrogen or an oxygen protecting group, with a compound of formula (IV):

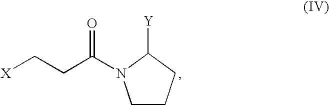

wherein X is halogen and Y is —C(O)NH₂ or —CN, removing the oxygen protecting group, R, when R is an oxygen protecting group, and converting —C(O)NH₂ to —CN when Y is —C(O)NH₂, and optionally crystallizing the compound of formula (I) from a solvent.

In some embodiments, when R of compound (III) is hydrogen, Y of compound (IV) is —C(O)NH₂. In other embodiments, when R of compound (III) is hydrogen, Y of compound (IV) is —CN.

The oxygen protecting group, R, can be any oxygen protecting group, which will prevent the reaction of oxygen in a compound of formula (III) during reaction with a compound of formula (IV), but which can later be removed from the intermediate that becomes the compound of formula (I) or formula (II). Suitable oxygen protecting groups are well-known to the skilled artisan and include, for example, silyl protecting groups, alkyl ethers, aralkyl ethers, ester protecting groups, and the like. In addition, any suitable conditions can be used to remove the oxygen protecting group, provided that the compound itself is not destroyed during the removal of the R protecting group. Suitable conditions for the removal of the oxygen protecting group are readily determined by one of ordinary skill, depending, in part, on the particular oxygen protecting group selected.

Preferably, X is a halogen. More preferably X is fluorine, chlorine, bromine, or iodine.

In accordance with the invention, any suitable solvent, or mixture of solvents, can be used for reacting a compound of formula (III) with a compound of formula (IV). In some embodiments, the reaction of a compound of formula (III) and a compound of formula (IV) is conducted in a solvent comprising at least one ether. Preferably, the ether is a C₂-C₆ cycloalkyl ether. In more preferred embodiments, the C₂-C₆ cycloalkyl ether is tetrahydrofuran. In a most preferred embodiment the solvent for the reaction of a compound of formula (III) with a compound of formula (IV) is tetrahydrofuran.

In other embodiments, the reaction of a compound of formula (III) and a compound of formula (IV) is conducted in a solvent comprising at least one ester. Preferably, the ester is a C₃-C₆ ester. In more preferred embodiments, the C₃-C₆ ester is selected from the group consisting of ethyl acetate, isopropyl acetate, and mixtures thereof.

In some embodiments, the reaction of a compound of formula (III) and a compound of formula (IV) is conducted in the presence of an inorganic base comprising an alkali metal carbonate. Illustrative alkali metals include, for example, lithium, sodium, potassium, rubidium, cesium, and francium. In a preferred embodiment, the alkali metal carbonate is cesium carbonate. In some embodiments, the reaction of a compound of formula (III) and a compound of formula (IV) is conducted in the presence of an inorganic base comprising a mixture of cesium carbonate and other alkali metal carbonates (e.g., potassium carbonate, sodium carbonate, sodium hydrogen carbonate, potassium hydrogen carbonate, and mixtures thereof).

In keeping with the invention, in preferred embodiments the reaction of a compound of formula (III) and a compound of formula (IV) is conducted in the absence of additives such as, for example, potassium iodide.

In some embodiments, the reaction of a compound of formula (III) and a compound of formula (IV) is conducted at a temperature of less than or about 50° C.

In keeping with the invention, any suitable conditions can be used to convert —C(O)NH₂ to —CN. Suitable conditions for the conversion of —C(O)NH₂ to —CN are known to the skilled artisan and include, for example, a dehydration reaction using P₂O₅, POCl₃ or cyanuric chloride. Suitable dehydration reagents are well-known in the art.

In preferred embodiments, the compound of formula (I) is crystallized from a solvent. In preferred embodiments, the crystallization solvent comprises at least one ketone. Preferably, the ketone is a C₃-C₇ ketone. More preferably, the C₃-C₇ ketone is methyl ethyl ketone.

In other preferred embodiments, the crystallization solvent comprises a mixture of at least one ether and at least one alcohol. In preferred embodiments, the mixture of an ether and an alcohol comprises a mixture of methyl tert-butyl ether and isopropyl alcohol.

The compound of formula (II) typically is formed during the synthesis of vildagliptin, in the process described herein. The compound of formula (II) is formed, for example, by the reaction of a compound of formula (IV) with the compound of formula (III), wherein an additional equivalent of compound (IV) is present such that the compound of formula (II) comprises at least two equivalents of a compound of formula (IV). In the process described above, the compound of formula (II) results from the removal of the oxygen protecting group, R, when present on the compound of formula (III) and by converting the amide (—C(O)NH₂) to the nitrile (—CN), when Y of compound (IV) is the amide.

It has been surprisingly discovered that the compound of formula (II) is useful as a reference marker for the analysis of vildagliptin or of compositions comprising vildagliptin. For example, the compound of formula (II) can be used as a reference marker in an analytical method to determine the chemical purity of the compound of formula (I). Suitable analytical methods are well-known to the skilled artisan. Illustrative analytical methods include, for example, high performance liquid chromatography (HPLC) and gas chromatography (GC), as well as, HPLC coupled to a Ultra-Violet Spectrometer (HPLC-UV) and HPLC coupled to a Mass Spectrometer (HPLC-MS). By way of illustration, the retention time of the compound of formula (II) can be determined in an analytical method (e.g., HPLC or GC), wherein the area of the corresponding peak can be compared to the peak area of another compound of interest in a mixture (e.g., vildagliptin prepared according to the present invention). The ratio of the peak areas can then be used to determine sample purity.

In keeping with the invention, the purity of a compound of formula (I) made in accordance with the novel process of the present invention, can be determined by monitoring the presence of the compound of formula (II). For example, the purity of the compound of formula (I) can be determined by a method comprising: a) identifying the retention times of compounds of formulae (I) and (II) using high performance liquid chromatography (HPLC); b) analyzing a sample comprising the compound of formula (I) using HPLC; and c) comparing the areas of the peaks associated with the retention times of compounds of formulae (I) and (II) in the sample, thereby determining the purity of the compound of formula (I). Similarly, the other analytical methods described above (e.g., HPLC-UV and HPLC-MS) can likewise be used to determine purity of a compound of formula (I).

Further, by way of illustration and not in limitation of the present invention, enantiomeric purity of the compound of formula (I) can be determined by a method comprising: a) identifying the retention times of the compound of formula (I) and the compound (2R)-1-[N-(3-hydroxytricyclo[3.3.1.1^{3,7}]dec-1-yl)glycyl]-2-pyrrolidinecarbonitrile using high performance liquid chromatography (HPLC); b) analyzing a sample comprising the compound of formula (I) using HPLC; and c) comparing the areas of the peaks associated with the retention times of the compound of formula (I) and the compound (2R)-1-[N-(3-hydroxytricyclo[3.3.1.1^{3,7}]dec-1-yl)glycyl]-2-pyrrolidinecarbonitrile in the sample, thereby determining the enantiomeric purity of the compound of formula (I).

Also, by way of example, the enantiomeric purity of the compound of formula (I) can be determined by a method comprising: a) identifying the retention times of the compound of formula (I) and the compound (2R)-1-[N-(3-hydroxytricyclo[3.3.1.1^{3,7}]dec-1-yl)glycyl]-2-pyrrolidinecarbonitrile using gas chromatography (GC); b) analyzing a sample comprising a compound of formula (I) using GC; and c) comparing the areas of the peaks associated with the retention times of the compound of formula (I) and the compound (2R)-1-[N-(3-hydroxytricyclo[3.3.1.1^{3,7}]dec-1-yl)glycyl]-2-pyrrolidinecarbonitrile in the sample, thereby determining the enantiomeric purity of the compound of formula (I).

In keeping with the invention, the chromatography is coupled to any suitable detector, as appropriate. For example, HPLC can be coupled to a ultraviolet detector or coupled to a mass spectrometer to aid in identification and quantification of compounds. In some embodiments, HPLC-UV is used to determine enantiomeric purity of vildagliptin of formula (I). In other embodiments, HPLC-MS is used to determine enantiomeric purity of vildagliptin of formula (I).

Vildagliptin made in accordance with the novel process described herein typically comprises the compound of formula (II), generally in an amount up to about 5%, by weight. Preferably, vildagliptin made in accordance with the novel process of the present invention comprises the compound of formula (II) in an amount up to about 1%, or up to about 2%, up to about 3%, or up to about 4%. More preferably, vildagliptin of the invention comprises the compound of formula (II) in an amount up to about 0.5%. In preferred embodiments, vildagliptin made in accordance with the process of the invention comprises the compound of formula (II) in an amount up to about 0.1% or even less than 0.1%. In even more preferred embodiments, vildagliptin made in accordance with the novel process described herein comprises trace amounts of the compound of formula (II).

Generally, the greater the purity of the vildagliptin made in accordance with the invention the more desirable the vildagliptin is for its ultimate intended use as a pharmaceutical. However, impurities of the nature of the compound of formula (II) are acceptable. Thus, in preferred embodiments, vildagliptin made by the process of the present invention comprises from 0.005% to 4%, 0.005% to 3%, 0.005% to 2%, or 0.005% to 1% of the compound of formula (II). More preferably, vildagliptin made in accordance with the invention comprises from 0.005% to 0.5% of the compound of formula (II). In a most preferred embodiment, vildagliptin made in accordance with the present invention comprises from 0.005% to 0.1% of the compound of formula (II). In some embodiments, the present invention provides vildagliptin having less than 1% of the compound of formula (II), preferably having less than 0.5% of the compound of formula (II) and more preferably having less than 0.1% of the compound of formula (II).

The invention provides (2S)-vildagliptin of formula (I) in high enantiomeric purity. For example, (2S)-vildagliptin made in accordance with the present invention comprises less than 0.01% of (2R)-1-[N-(3-hydroxytricyclo[3.3.1.1^{3,7}]dec-1-yl)glycyl]-2-pyrrolidinecarbonitrile (i.e., (2R)-vildagliptin).

Another additional aspect of the present invention provides vildagliptin having less than 1% of individual impurities, preferably having less than 0.5% of individual impurities, and more preferably having less than 0.1% of individual impurities.

The present invention also provides compositions comprising vildagliptin of formula (I) made in accordance with the process described herein. The vildagliptin can include the compound of formula (II) in an amount as described heretofore. Preferably compositions comprising (2S)-vildagliptin of formula (I) comprise less than 0.01%, less than 0.1%, or less than 0.5% of (2R)-1-[N-(3-hydroxytricyclo[3.3.1.1^{3,7}]dec-1-yl)glycyl]-2-pyrrolidinecarbonitrile (i.e., (2R)-vildagliptin).

Compositions of the invention can be any suitable form (e.g., solid, liquid, or gas) and can include any suitable excipient as appropriate. In a preferred embodiment, a composition in accordance with the invention is a solid. In particular preferred embodiments, compositions of the invention are a powder. Further, compositions of the invention can be formulated into any suitable pharmaceutical dosage form as appropriate. Suitable pharmaceutical dosage forms are known to the skilled artisan and include, for example, tablets, capsules, pills, syrups, patches, lozenges, aerosols, emulsions, and the like.

The following examples further illustrate the invention but, of course, should not be construed as in any way limiting its scope.

### EXAMPLE 1

This example illustrates a HPLC method for identifying vildagliptin of formula (I) using an Agilent 1100 Series HPLC in accordance with the invention.

HPLC was performed using a NUCLEOSIL 120 C₁₈ column (25 mm×0.4 mm, 10 μm, Scharlab, S.L.). The mobile phase was a two-component system comprising A) 0.02 M potassium dihydrogen phosphate adjusted to pH 8 and B) acetonitrile. Samples were prepared having a concentration of 2 mg/mL. Ten microliters of sample were injected onto the column at a temperature of 25° C. and a flow rate of 1.0 mL/min. The detector was set to monitor 210 nm.

The elution profile is provided in Table 1.

TABLE 1

| time (min) | mobile phase A (% v/v) | mobile phase B (% v/v) |
|---|---|---|
| 0 | 90 | 10 |
| 5 | 90 | 10 |
| 25 | 10 | 90 |
| 45 | 10 | 90 |
| 50 | 90 | 10 |
| 60 | 90 | 10 |

The retention time for vildagliptin using these conditions was 13.7 min.

### EXAMPLE 2

This example illustrates a HPLC method for identifying vildagliptin of formula (I) in accordance with the invention.

HPLC was performed using a Synergi Fusion RP80A column (250 mm×4.6 mm I.D., 4 μm) at 30° C. The mobile phase was a two-component system. Component A was prepared from 1.36 g of KH₂PO₄ salt in 1000 mL of water, adjusting the pH to 7.3 using KOH, and filtering through a 0.22 μm nylon filter under vacuum; component B was acetonitrile.

The chromatograph was programmed as follows: initial 0-9 min. isocratic 85% mobile phase A, 9-40 min. linear gradient to 60% mobile phase A, 40-50 min. isocratic 60% mobile phase A, 50-55 min. linear gradient to 85% mobile phase A and 55-60 min. equilibration to 85% mobile phase A.

Samples were prepared having a concentration of 6 mg/mL in a mixture of A:B (6:4). Twenty microliters of sample were injected onto the column at a flow rate of 1.0 mL/min. The chromatograph was equipped with a detector monitoring 210 nm.

### EXAMPLE 3

This example illustrates the synthesis of a compound of formula (IV) in accordance with the invention.

Synthesis of 1-chloroacetyl-2-cyanopyrrolidine. A 250 mL reactor with thermometer, condenser and magnetic stirring was charged with i-PrOAc (41 mL), dry DMF (5 mL) and chloroacetyl chloride (19.5 g, 173 mmol) under an inert atmosphere. The resulting solution was cooled to 15° C. and a solution of L-prolinamide (17.1 g, 150 mmol) in dry DMF (48 mL) was slowly added (IT≦35° C.). The reaction mixture was stirred 2 additional hours at 35° C. to obtain complete conversion. The internal temperature was adjusted to 25° C. and cyanuric chloride (13.8 g, 75 mmol) was added in portions (IT≦35° C.). The mixture was stirred for 45 minutes, poured into 200 mL of water and extracted with EtOAc (3×200 mL). The organic phase was washed with 5% aqueous NaHCO₃ (2×200 mL), dried over Na₂SO₄, filtered, and the solvents were evaporated under vacuum. The resulting oil was crystallized in 65 mL of isopropanol to obtain 17.16 g of 1-chloroacetyl-2-cyanopyrrolidine (66% yield).

### EXAMPLE 4

This example illustrates the synthesis of the compound of formula (II) in accordance with an embodiment of the invention.

Synthesis of (2S,2′S)-1,1′-[[(3-hydroxytricyclo[3.3.1.1^{3,7}]dec-1-yl)imino]bis(1-oxo-2,1-ethanediyl)]di(2-pyrrolidinecarbonitrile). A 50 mL round bottom flask with magnetic stirring was charged with THF (15 mL), powdered K₂CO₃ (3.3 g, 24 mmol), 3-amino-1-adamantanol (1 g, 6 mmol), 1-chloroacetyl-2-cyanopyrrolidine (2.6 g, 15 mmol) and KI (50 mg, 0.3 mmol). The resulting slurry was heated to reflux for 5 hours. The suspension was cooled down, filtered at room temperature and the solids washed with 5 mL of THF. Solvents were distilled off under vacuum and the resulting crude was recrystallized in 15 mL of isopropanol to obtain 1.9 g of the compound of formula II (72% yield): mp 181-183° C.; IR (KBr) 3420, 2920, 2906, 2880, 2851, 2239, 1650 1450, 1424, 1403, 1311, 1003 cm⁻¹; ¹H-NMR (DMSO-d6) 1.30-1.67 (m, 12H), 1.75-2.36 (m, 10H), 3.10-3.29 (m, 1.3H), 3.38-3.84 (m, 6.7H), 4.40-4.51 (m, 1H), 4.57-4.70 (m, 1.5H), 5.95-6.07 (m, 0.4H); ¹³C-NMR (DMSO-d6, 80° C.) 24.4, 28.9, 29.9, 34.5, 37.8, 43.9, 45.3, 45.9, 46.8, 49.9, 57.5, 67.5, 118.8, 170.0; MS (ESI+) 440 (M⁺+1); [α]_{D}²⁵=111 (c 1.0, MeOH).

### EXAMPLE 5

This example illustrates the synthesis of the compound of formula (I) in accordance with the invention.

A 250 mL reactor with thermometer, condenser and magnetic stirring was charged with THF (90 mL), powdered K₂CO₃ (21.3 g), 3-amino-1-adamantanol (9 g), 1-chloroacetyl-2-cyanopyrrolidine (8.85 g) and KI (0.43 g). The resulting slurry was heated to reflux until complete conversion by TLC (approx. 2 h). The warm suspension was filtered and the solids washed with 30 mL of THF. Solvents were distilled off under vacuum to obtain 16.4 g of a solid. This solid was suspended in 52 mL of MEK and heated to reflux. The resulting clear solution was allowed to cool and the product crystallized as a white solid. The slurry was stirred at 0° C. for 1 hour, filtered, washed with a cold mixture of MEK/MeOBu^{t} (1/1) and dried under vacuum to obtain 10.3 g (66% yield). HPLC analysis showed vildagliptin and compound (II) in a 99.7:0.3 ratio. mp 150° C.; ¹H-NMR (CDCl₃) 1.45-1.73 (m, 12H), 1.81 (brs, 2H), 2.03-2.45 (m, 6H), 3.32-3.76 (m, 4H), 4.70-4.81 (m, 0.8H), 4.83-4.91 (m, 0.2H); ¹³C-NMR (CDCl₃ ) 22.8, 25.0, 29.9, 30.7, 32.3, 35.07, 35.12, 41.07, 41.14, 41.2, 41.3, 43.4, 44.4, 45.5, 46.3, 46.5, 46.6, 49.9, 53.4, 53.7, 69.5, 118.2, 170.6; MS (ESI+) 304 (M⁺+1); [α]_{D}²⁵=−85 (c 9.73, MeOH).

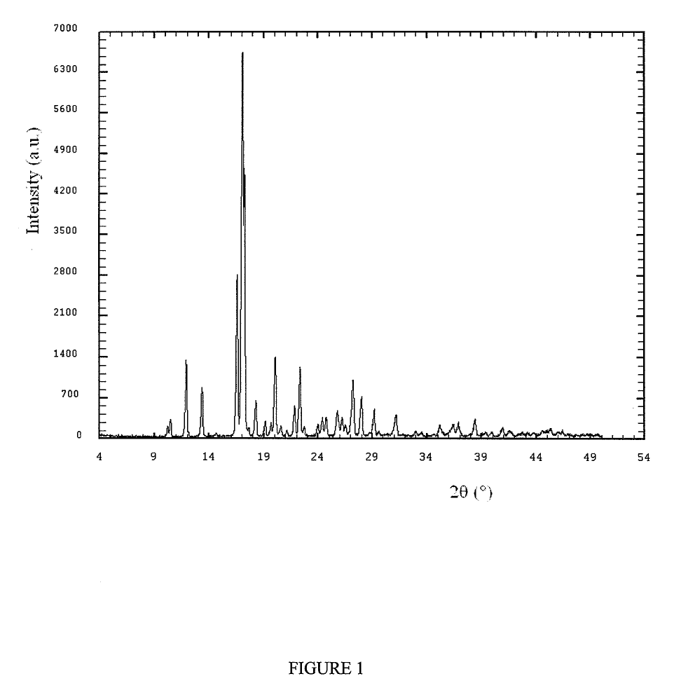

The vildagliptin obtained had the powder X-ray diffractogram depicted in FIG. 1.

### EXAMPLE 6

This example illustrates a method for determining the enantiomeric purity of compound of formula (I) by means of HPLC-UV in accordance with an embodiment of the invention.

The chromatographic separation was carried out using a Daicel CHIRALPAK® IC column (4.6 mm×250 mm, 5 μm). The mobile phase was prepared by mixing 100 mL of ethanol with 0.1 mL of diethylamine.

Samples were prepared by dissolving 10 mg of sample in 1 mL of ethanol. Ten microliters were injected onto the column at 20-25° C. at a flow rate of 0.5 mL/min. The chromatograph was equipped with a detector monitoring 210 nm.

### EXAMPLE 7

This example illustrates a method for determining the enantiomeric purity of compound of formula (I) by means of HPLC-MS in accordance with an embodiment of the invention.

Samples were prepared by dissolving 15 mg of sample in 1 mL of ethanol. The mobile phase was 100% ethanol. Ten microliters were loaded onto the HPLC.

An Agilent 1200 series HPLC system was equipped with a 210 nm detector and was coupled to an Applied Biosystems API 2000 LC/MS/MS mass spectrometer. The flow rate was 0.5 mL/min at 20-25° C., the flow was splitted and 0.2 mL/min entered in the spectrometer. Electrospray negative mode was used. The conditions of the source were DP=−80, FP=−100, EP=−10, CUR=10, IS=−4500, TEM=300, GS1=40 and GS2=60. The centered mass mode was used (centered in 302 amu, width was 4 amu and time was 3 seconds).

### EXAMPLES 8-23

These examples illustrate the synthesis of the compound of formula (I) in accordance with embodiments of the invention. In addition, these examples illustrate a method for determining the purity of a compound of formula (I) in accordance with the invention.

The following general procedure was used: into a 10 or 25 mL reactor were charged: 0.51 g (2.95 mmol) of 1-chloroacetyl-2-cyanopyrrolidine, 0.57 g (3.40 mmol) of aminoadamantanol, 3.40 mmol of potassium or cesium carbonate and, when indicated, 5 mg (0.03 mmol) of KI. Finally, 5 mL of solvent was also added. The mixture was heated at reflux (or at the temperature indicated in the table) for approximately 14 h, then cooled to room temperature filtered to remove the inorganic salts, which were washed with acetonitrile, the organic phases were collected and evaporated to dryness. A sample was collected for HPLC analysis. After crystallization from a mixture of IPA and MTBE the typical overall yield was about 60-70%.

The results are summarized in Table 2. The HPLC purity was measured according to Example 2.

TABLE 2

| | | M₂CO₃ | | | Ratio Compound |
|---|---|---|---|---|---|
| | | (M = | | | I/Compound II |
| Exam- | | Cs | | Temperature | (relative % area |
| ple | Solvent | or K) | KI | (° C.) | HPLC) |
| 8 | THF | K | Yes | 66 | 4.97 |
| 9 | THF | Cs | Yes | 66 | 4.39 |
| 10 | THF | Cs | No | 66 | 12.13 |
| 11 | MeCN | Cs | Yes | 70 | 12.00 |
| 12 | MeCN | Cs | No | 70 | 8.43 |
| 13 | THF | Cs | No | 45 (24 h) | 18.4 |
| 14 | THF | Cs | No | 66 | 11.15 |
| 15 | EtOAc | Cs | No | 70 | 20.77 |
| 16 | THF | K | No | reflux | 6.87 |
| 17 | i-PrOAc | Cs | No | 70 | 17.77 |
| 18 | MeCN/i- | Cs | No | 70 | 13.64 |
| | PrOAc(1:1) | | | | |
| 19 | EtOAc | Cs | No | 45 | 34.90 |
| 20 | EtOAc | K | No | 45 | 16.43 |
| 21 | EtOAc | Cs | No | 45 | 29.04 |
| 22 | i-PrOAc | K | No | 45 | 11.68 |
| 23 | i-PrOAc | Cs | No | 45 | 39.91 |

These results demonstrate that vildagliptin of formula (I) having a high purity can be prepared using processes in accordance with the invention. Further, these results demonstrate that the purity of a compound of formula (I) can be determined using methods in accordance with the invention.

### EXAMPLE 24

This example illustrates the synthesis of the compound of formula (I) in accordance with embodiments of the invention.

Into a 100 mL rounded reaction vessel were charged 3 g (17.37 mmol) of 1-chloroacetyl-2-cyanopyrrolidine, 3.22 g (19.82 mmol) of 1-amino-3-adamantanol, 2.78 g (20.1 mmol) of potassium carbonate, and 30 mL isopropyl acetate. The mixture was refluxed for 4 h, cooled to room temperature, and the salts were filtered and washed with acetonitrile. The mother liquors were evaporated to dryness to obtain an oil which was aged in MEK from which a white solid crystallizes at 0-5° C. The solid was filtered washing the cake with MEK and dried at 40° C. in a vacuum oven until constant weight.

Yield: 36%. Assay: 99.21%. HPLC purity: 97.55% of vildagliptin (measured according to Example 2). HPLC chiral purity: more than 99.99% of vildagliptin (measured according to Example 7).

These results demonstrate that a compound of formula (I) comprising less than 0.01% of (2R)-1-[N-(3-hydroxytricyclo[3.3.1.1^{3,7}]dec-1-yl)glycyl]-2-pyrrolidinecarbonitrile (i.e., (2R)-vildagliptin).

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

1. The compound (2S)-1-[N-(3-hydroxytricyclo[3.3.1.1³⁷]dec-1-yl)glycyl]-2-pyrrolidinecarbonitrile of formula (I): 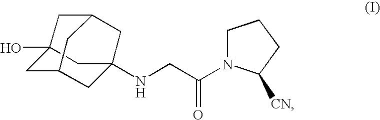
comprising (2S,2′S)-1,1′-[[(3 -hydroxytricyclo[3.3.1.1^{3,7}]dec-1-yl)imino]bis(1-oxo-2,1-ethanediyl)]di(2-pyrrolidinecarbonitrile) of formula (II): 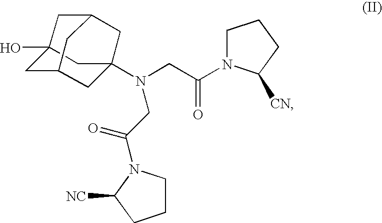
in an amount up to about 5%.

2. The compound (2S,2′S)-1,1′-[[(3-hydroxytricyclo[3.3.1.1^{3,7}]dec-1-yl)imino]bis(1-oxo-2,1-ethanediyl)]di(2-pyrrolidinecarbonitrile) of formula (II) and salts thereof:

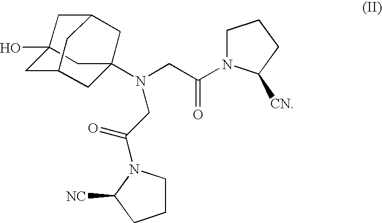

3. The compound (2S)-1-[N-(3-hydroxytricyclo[3.3.1.1^{3,7}]dec-1-yl)glycyl]-2-pyrrolidinecarbonitrile of formula (I): 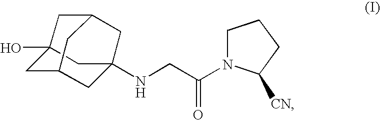
comprising less than 0.01% of (2R)-1-[N-(3-hydroxytricyclo[3.3.1.1^{3,7}]dec-1-yl)glycyl]-2-pyrrolidinecarbonitrile.

4. A process for preparing (2S)-1-[N-(3-hydroxytricyclo[3.3.1.1^{3,7}]dec-1-yl)glycyl]-2-pyrrolidinecarbonitrile of formula (I): 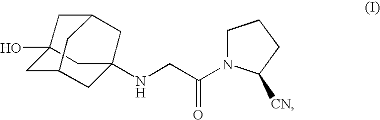
comprising reacting a compound of formula (III): 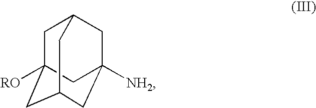
wherein R is hydrogen or an oxygen protecting group, in a solvent comprising at least one ether, with a compound of formula (IV): 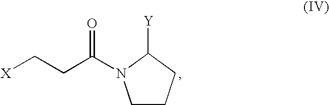
wherein X is halogen and Y is —C(O)NH₂ or —CN; removing the oxygen protecting group, R, when R is an oxygen protecting group; and converting —C(O)NH₂ to —CN when Y is —C(O)NH₂; and optionally crystallizing the compound of formula (I) from a solvent comprising at least one ketone; with the proviso that when R of compound (III) is hydrogen, Y of compound (IV) is —C(O)NH₂.

5. The process of claim 4, wherein the ether is a C₂-C₆ cycloalkyl ether.

6. The process of claim 5, wherein the C₂-C₆ cycloalkyl ether is tetrahydrofuran.

7. The process of claim 4, wherein the ketone is a C₃-C₇ ketone.

8. The process of claim 7, wherein the C₃-C₇ ketone is methyl ethyl ketone.

9. A process for preparing (2S)-1-[N-(3-hydroxytricyclo[3.3.1.1^{3,7}]dec-1-yl)glycyl]-2-pyrrolidinecarbonitrile of formula (I): 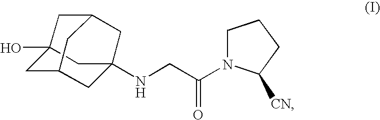
comprising reacting a compound of formula (III): 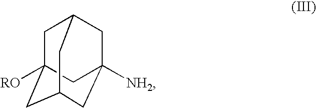
wherein R is hydrogen or an oxygen protecting group, with a compound of formula (IV): 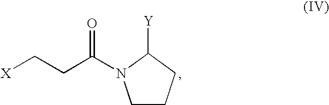
wherein X is halogen and Y is —C(O)NH₂ or —CN, in the presence of an inorganic base comprising an alkali metal carbonate, in a solvent comprising at least one ester, in the absence of potassium iodide, and at a temperature of less than or about 50° C.; removing the oxygen protecting group, R, when R is an oxygen protecting group; converting —C(O)NH₂ to —CN when Y is —C(O)NH₂; and optionally crystallizing the compound of formula (I) from a solvent comprising at least one ketone or a mixture of at least one ether and at least one alcohol; with the proviso that when R of compound (III) is hydrogen, Y of compound (IV) is —C(O)NH₂.

10. The process of claim 9, wherein the mixture of at least one ether and at least one alcohol is a mixture of methyl tert-butyl ether and isopropyl alcohol.

11. The process of claim 9, wherein the alkali metal carbonate is cesium carbonate.

12. The process of claim 11, wherein the inorganic base comprising cesium carbonate further comprises an inorganic base selected from the group consisting of potassium carbonate, sodium carbonate, sodium hydrogen carbonate, potassium hydrogen carbonate, and mixtures thereof.

13. The process of claim 9, wherein the ester is a C₃-C₆ ester.

14. The process of claim 13, wherein the C₃-C₆ ester is selected from the group consisting of ethyl acetate, isopropyl acetate, and mixtures thereof.

15. The process of claim 9, wherein the ketone is a C₃-C₇ ketone.

16. The process of claim 15, wherein the C₃-C₇ ketone is methyl ethyl ketone.

17. A process for preparing (2S,2′S)-1,1′-[[(3-hydroxytricyclo[3.3.1.1^{3,7}]dec-1-yl)imino]bis(1-oxo-2,1-ethanediyl)]di(2-pyrrolidinecarbonitrile) of formula (II): 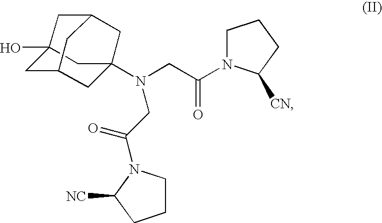
comprising reacting a compound of formula (III): 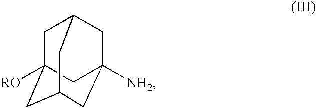
wherein R is hydrogen or an oxygen protecting group, with a compound of formula (IV): 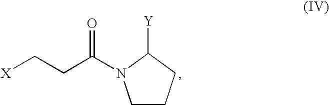
wherein X is halogen and Y is —C(O)NH₂ or —CN; removing the oxygen protecting group, R, when R is an oxygen protecting group; and converting —C(O)NH₂ to —CN when Y is —C(O)NH₂.

18. A method for determining the purity of the compound of formula (I) comprising: a) identifying the retention times of compounds of formulae (I) and (II) using high performance liquid chromatography (HPLC); b) analyzing a sample comprising a compound of formula (I) using HPLC; and c) comparing the areas of the peaks associated with the retention times of compounds of formulae (I) and (II) in the sample, thereby determining the purity of the compound of formula (I).

19. A method for determining the enantiomeric purity of the compound of formula (I) comprising: a) identifying the retention times of the compound of formula (I) and the compound (2R)-1-[N-(3-hydroxytricyclo[3.3.1.1^{3,7}]dec-1-yl)glycyl]-2-pyrrolidinecarbonitrile using high performance liquid chromatography (HPLC); b) analyzing a sample comprising the compound of formula (I) using HPLC; and c) comparing the areas of the peaks associated with the retention times of the compound of formula (I) and the compound (2R)-1-[N-(3-hydroxytricyclo[3.3.1.1^{3,7}]dec-1-yl)glycyl]-2-pyrrolidinecarbonitrile in the sample, thereby determining the enantiomeric purity of the compound of formula (I).

20. A method for determining the enantiomeric purity of the compound of formula (I) comprising: a) identifying the retention times of the compound of formula (I) and the compound (2R)-1-[N-(3-hydroxytricyclo[3.3.1.1^{3,7}]dec-1-yl)glycyl]-2-pyrrolidinecarbonitrile using high performance liquid chromatography (HPLC) coupled with a Ultra-Violet Spectrometer (UV); b) analyzing a sample comprising the compound of formula (I) using HPLC; and c) comparing the areas of the peaks associated with the retention times of the compound of formula (I) and the compound (2R)-1-[N-(3-hydroxytricyclo[3.3.1.1^{3,7}]dec-1-yl)glycyl]-2-pyrrolidinecarbonitrile in the sample, thereby determining the enantiomeric purity of the compound of formula (I).

21. A method for determining the enantiomeric purity of the compound of formula (I) comprising: a) identifying the retention times of the compound of formula (I) and the compound (2R)-1-[N-(3-hydroxytricyclo[3.3.1.1^{3,7}]dec-1-yl)glycyl]-2-pyrrolidinecarbonitrile using high performance liquid chromatography (HPLC) coupled with a Mass Spectrometer (MS); b) analyzing a sample comprising the compound of formula (I) using HPLC; and c) comparing the areas of the peaks associated with the retention times of the compound of formula (I) and the compound (2R)-1-[N-(3-hydroxytricyclo[3.3.1.1^{3,7}]dec-1-yl)glycyl]-2-pyrrolidinecarbonitrile in the sample, thereby determining the enantiomeric purity of the compound of formula (I).

22. A composition comprising a compound of claim 1.

23. A composition comprising a compound of claim 3.
